(19) United States
(12) Patent Application Publication
(10) Pub. No.: US 2023/0243823 A1
(43) Pub. Date: Aug. 3, 2023

(54) BIOMARKER SENSOR-BASED DETECTION OF DISEASE-SPECIFIC BIOMARKERS

(71) Applicant: FORSCHUNGSZENTRUM JÜLICH GMBH, Juelich (DE)

(72) Inventors: Raffaele VELOTTA, Napoli (IT); Dirk MAYER, Frechen (DE); Bartolomeo DELLA VENTURA, Maddaloni (Caserta) (IT); Antonio MINOPOLI, Casalnuovo di Napoli (IT); Bohdan LENYK, Aachen (DE)

(21) Appl. No.: 17/999,190

(22) PCT Filed: Jun. 7, 2021
(86) PCT No.: PCT/EP2021/065114

§ 371 Date: Nov. 17, 2022

(30) Foreign Application Priority Data
Jun. 9, 2020 (DE) 10 2020 115 332.5

(51) Int. Cl. G01N 33/569; G01N 33/543; G01N 33/53

(52) U.S. Cl. CPC G01N 33/56905; G01N 33/54373; G01N 33/54346; G01N 33/5308; G01N 2469/10; G01N 2333/904

(57) ABSTRACT

Biomarker detection sensors for detecting disease-specific biomarkers, kits based thereon, methods for determining disease-specific biomarkers in body fluids and uses are presented. The sensors include a substrate having bound thereon metal nanoparticles arranged in branched two-dimensional structures and/or a two-dimensional lattice, and anti-biomarker receptors bound to the metal nanoparticles.

The present invention relates to biomarker detection sensors for the detection of disease-specific biomarkers, kits based on these sensors, methods for the determination of disease-specific biomarkers in body fluids and uses.

Fluorescence-based techniques are among the most widely used methods in the fields of biotechnology, life sciences and (bio-)medical research. Fluorescent samples and fluorescence-labelled bioreceptors are extensively used in optogenetic studies, cytogenetic assays, multicolour fluorescent in situ hybridisation (mFISH), in bioimaging analysis, for the observation of both the location as well as the movement of cells or subcellular elements, in the study of molecular dynamics, as well as in fluoroimmunoassays for the detection of molecular biomarkers, as enzyme-linked immunosorbent assays (ELISA). However, the application of fluorescence methods for sensory processing is severely limited by the low yields of many fluorescent dyes and the occurrence of signal interference. In particular, the detection of very low analyte concentrations is still one of the main problems. Many attempts have been made to amplify the fluorescence signals to enable their applicability for ultrasensitive fluoroimmunoassays.

To overcome this challenge—without the use of expensive equipment, specific or toxic reagents, or to have to do significant modifications to well established fluorescence-based assays—plasmonic nanostructures have gained attention in recent years. This is due to the ability of plasmonic nanostructures to influence the spectral properties of nearby fluorescent dyes. This influence depends strongly on the spectral overlap between the fluorescent dye and the plasmonic absorption, as well as on the distance z between fluorophore and nanostructure. In particular, due to the Foerster resonance energy transfer mechanism (FRET) (i.e. dipole-dipole interactions between the surface plasmon and the fluorophore), a strong fluorescence enhancement (FE) is achieved for small z (<10 nm) when the plasmonic absorption overlaps the excitation peak of the fluorophore (excitation coupling). In contrast, for large z, the weak coupling leads to negligible fluorescence enhancement. Contrary to that, the overlap with the fluorophore emission peak (emission coupling) leads to a strong fluorescence enhancement at large z (>10 nm), due to the enhancement of the fluorophore radiation rate by the Purcell effect, whereas a progressive decrease of the fluorescence enhancement occurs at smaller z due to the increasing fluorescence quenching via non-radiative losses in the metallic nanostructure. In the case where the plasmon absorption overlaps both the excitation as well as the emission peaks of the fluorophore (combined mode), a very large fluorescence enhancement occurs at an optimal z of about 10-15 nm; a strong quenching occurs at short z, whereas restoration of normal fluorescence conditions (i.e., in the absence of a plasmonic structure) occurs at longer distances due to the weaker plasmon-fluorophore coupling.

Two-dimensional (2D) arrays of metallic nanostructures are particularly suitable as plasmon enhanced fluorescence (PEF)-based biosensing platforms. So far, various fluorescence enhancers such as gold microislands or arrays of metallic nanoobjects (e.g. nanoparticles, nanotubes, nanotriangles, nanocrystals, nanoantennas and resonant nanocavities) have been tried to push the detection limit in fluorescence-based assays further down. Despite the great improvements with respect to the achievable limits of detection (LOD) reaching down to the femto-molar level, the need for trained personnel, expensive technologies, time-consuming procedures, low versatility, poor scalability and low signal-to-noise ratio represent limiting factors for routine testing, point-of-care analysis and large-scale use of PEF-based fluoroassays.

The development of plasmonic biosensors that combine reliability and ease of use is still a challenge.

The object of the present invention was to provide possibilities for the highly sensitive determination of disease-specific biomarkers that no longer exhibit the problems of the prior art.

Further objects arise for the person skilled in the art when considering the following description and the claims.

These and further objects, which become apparent to the person skilled in the art upon consideration of the present description, are solved by the subject matter outlined in the independent claims.

Particularly advantageous and preferred subject matter will be apparent from the dependent claims and the following description.

In the context of the present invention, it has been found that 2D frameworks of ordered and periodic gold nanoparticles (AuNPs) fabricated via self-assembled diblock copolymer nanolithography are an effective way to overcome most of the identified problems of the prior art. The cheap and scalable preparation and the easy tunability of their plasmonic properties, by adjusting the AuNP size and the interparticle distance by simply varying the lengths of hydrophilic and hydrophobic part of the copolymers, respectively, are the main strengths of such a PEF platform. The plasmonic behaviour of a 2D framework of well-ordered AuNPs is closely related to the ratio R between the nanoparticle diameter D and the interparticle distance d. When each nanoparticle is in close proximity to its neighbours (i.e. when R becomes >2/3), a long-range collective oscillation called delocalised surface plasmon (DSP) is obtained by the interaction between the localised surface plasmons (LSPs). Such a collective effect becomes negligible the more R becomes <2/3; in this case, the plasmonic response of the 2D framework is well described by a system of decoupled LSPs.

In the context of the present invention, room temperature means a temperature of 293.15 Kelvin, i.e. 20° C.

Unless further specified, atmospheric pressure (1013 mbar_{absolute}) and room temperature prevail in all reactions and procedures.

In the present invention particularly a plasmon enhanced fluoroscence immunosensor for the determination and detection of Plasmodium falciparum lactate dehydrogenase (P [symbol] DH)—a malaria marker—in whole blood samples is described.

In the context of the present invention, analyte determination is achieved using oriented antibodies immobilised in a close-packed configuration by photochemical immobilisation technique (PIT). The combination of BCMN (Block Copolymer Micelle Nanolithography) and PIT allows maximum control over the nanoparticle size and lattice constant, as well as the distance of the fluorophore from the sensor surface. Due to these characteristics, in some embodiments the arrangements of the present invention achieve a detection limit of less than 1 pg/ml (<30 fM) with very high specificity without any pre-treatment of the samples; this is achieved according to the invention, in particular in the embodiments where the gold nanoparticles form a two-dimensional lattice in the form of honeycombs, i.e. the gold nanoparticles are preferably arranged hexagonally or hexagonally close. This detection limit is several orders of magnitude lower than what is achieved in malaria rapid tests or even commercial ELISA kits.

In other embodiments that are not fully optimised for maximum LOD (Limit Of Detectability), for example when sample concentrations are in the range of nM or μM, these sensitivities may not be necessary. However, in these embodiments, which are particularly characterised by the use of multiple fluorophores, confirmation can be achieved by redundant, second signals or detection of a second antigen and the detection, for example, down to 50 pM for a green fluorescent fluorophore and down to 260 fM for a red fluorescent fluorophore. By the combination of a fluorophore that allows low LODs with a fluorophore that has a high LOD, the concentration range in which the biomarker can be detected can be broadened.

Due to their overall dimensions, simplicity of use and high analytical throughputs, the arrangements of the present invention, i.e. the biomarker detection sensors can be used as substrates in multititer plates. The efficiency is significantly improved over conventional fluorescence immunoassays.

Subject matter of the present invention is a biomarker detection sensor for the detection of disease-specific biomarkers comprising or consisting of a substrate having bound thereon metal nanoparticles, in particular gold nanoparticles, arranged in branched two-dimensional structures and/or a two-dimensional lattice, and anti-biomarker receptors bound to the metal nanoparticles, in particular gold nanoparticles.

In broad embodiments of the present invention, various possibilities are conceivable by means of which the detection of the specific biomarkers can be indicated. For example, this is possible by designing the detection sensors as microbalances, by means of which specifically bound biomarkers can then be balanced, i.e. detection takes place via an increase in the weight of the sensor.

In preferred embodiments of the present invention, however, detection is carried out by means of fluorescence analyses, since these are particularly easy to implement in continuous operation.

To this end, in embodiments of the present invention, the electronic structure of the biomarker detection sensors is modified by the attachment of the disease-specific biomarkers in such a way that they are measurable in their fluorescence emission or extinction spectrum.

Within the scope of the present invention, virtually any disease-specific biomarker can be detected based on the principle of the invention, it is only necessary that the anti-biomarker receptors bound to the gold nanoparticles are sufficiently specific for these corresponding biomarkers.

The substrates of the biomarker detection sensors according to the present invention can be any substrates on which the nanoparticles can be deposited. In embodiments, these are known assay wells, surfaces based on polyacrylates (e.g. Plexiglas®), polystyrene or glass surfaces. In principle, all substrates that are stable under the experimental conditions (including production), are transparent to excitation and fluorescence in the respective wavelength range examined/applied and do not exhibit intrinsic fluorescence are also suitable.

The metal nanoparticles to be deposited on the substrates can, in principle, be selected on any material that is non-corrosive under fabrication and examination conditions. In variants of the present invention, the materials may be selected from the group consisting of noble metals, in particular gold, silver, platinum, copper, chromium, alloys thereof and Janus particles of these metals.

It is particularly advantageous and preferred if the metal nanoparticles are gold nanoparticles.

Preferably, in the context of the present invention, the disease to be detected is malaria. In particular, the disease-specific biomarker is then Plasmodium falciparum L-lactate dehydrogenase (P<DH).

Accordingly, in preferred embodiments of the present invention, the anti-biomarker receptors bound to the gold nanoparticles are anti-pLDH antibodies.

According to the invention, these anti-pLDH antibodies are fixed to the gold nanoparticles, i.e. bound thereto. Thus, biomarker detection sensors of the aforementioned type are encompassed by the present invention, wherein the anti-biomarker receptors are anti-pLDH antibodies bound to the gold nanoparticles via thiol groups.

In particular embodiments of the present invention, these anti-pLDH antibodies are anti-pLDH antibodies pre-treated with UV radiation.

By means of UV radiation as a pre-treatment, it is possible to modify the anti-pLDH antibodies in such a way that thiol groups become available by cleavage of disulphide bridges.

In the case of in anti-pLDH antibodies, UV irradiation causes a selective photoreduction of the disulphide bridges in specific cysteine-cysteine/tryptophan triads (where the presence of the tryptophan activates the disulphide bridge in this triad, so to speak). The cleavage of these Cys-Cys bonds in both antibody fragments leads to a total of four free thiol groups, two of which can interact with the surface of the gold nanoparticles to form a covalent bond.

In addition, by this type of binding it is achieved that the specific region of the anti-pLDH antibody to which the P<DH binds is oriented away from the gold nanoparticles into the space and thus becomes readily accessible to the P<DH.

In preferred embodiments of the present invention, the UV treatment is carried out for 30 seconds at a radiation power of 1 watt/cm² and at a wavelength of 254 nm or at a radiation power of 6 watts/cm² and at a wavelength of 254 nm.

Although the present invention is essentially directed to gold nanoparticles, it should be noted that the present invention is in principle also conceivable with other metal atoms.

Furthermore, the present invention encompasses biomarker detection sensors in which the ratio R of the diameter D of the nanoparticles to the distance d between the individual gold nanoparticles (edge-to-edge distance) in the two-dimensional lattice is >2.3. Preferably, this ratio is between 2.3 and 3.5, and particularly preferably 2.5 or 3.0.

In one alternative, it is particularly preferred in the context of the present invention if the gold nanoparticles form a two-dimensional lattice in the form of honeycombs, i.e. the gold nanoparticles are preferably arranged hexagonally or hexagonally close. In another alternative, it is particularly preferred in the context of the present invention if the gold nanoparticles form a two-dimensional branched structure, i.e. the gold nanoparticles are preferably arranged in branched band-like structures, with only a few particles (2-4) having a dense packing at any one time.

In the context of the present invention, a third alternative is particularly preferred in which the gold nanoparticles form a combined two-dimensional structure in which both parts of the structure form lattices in the form of honeycombs, i.e. the gold nanoparticles in these parts are preferably arranged hexagonally or hexagonally close, and at the same time parts of the structure form two-dimensional branched structures, i.e. the gold nanoparticles in these parts are preferably arranged in branched, band-like structures whereby always only a few particles (2-4) have a close packing and are otherwise randomly distributed. In this alternative, the close-packed nanoparticles in the band-like structures can cause absorptions at 680 nm (delocalised surface plasmons) and the particles that have close distances to only a few neighbours can cause absorptions at about 530 nm (localised surface plasmons LSPR).

In preferred embodiments of the present invention, the diameter D of the nanoparticles of the biomarker detection sensors is between 40 and 60 nm, preferably between 44 and 55 nm, particularly preferably between 46 and 52 nm, and especially preferably about 48 or about 50 nm. In other embodiments, the diameter D of the nanoparticles of the biomarker detection sensors is between 50 and 70 nm, preferably between 55 and 65 nm particularly preferably 60 nm.

The distance d between the individual gold nanoparticles (the edges of the individual particles) of the biomarker detection sensors is preferably between 16 and 24 nm, more preferably between 17 and 22 nm, particularly preferably between 18 and 21 nm and especially preferably at 19 or 20 nm.

The sizes were determined by scanning electron microscopy. An example of a usable microscope is a Zeiss® Leo 1550 VP.

With regard to the diameter D of the nanoparticles, it should be noted that the corresponding data refer to mean diameters; naturally, the effective diameters in these orders of magnitude scatter a little around the correspondingly named specific value. This in turn also means that the distance d between the particles scatters around a central value. This is known to the person skilled in the art.

For a diameter D of 47.67 nm, for example, the deviation in embodiments is plus/minus 0.14 nm.

For an interparticle distance (measured centre to centre) of 68.47 nm, for example, the deviation in embodiments is plus/minus 0.19 nm.

The specific structure of the AuNP on the substrate can be controlled in the fabrication of the biomarder detection sensors of the present invention.

A simple variation to switch between hexagonal structures and branched structures is to regulate the deposition speed; if the speed is increased above a certain value, the particles do not have enough time to arrange ideally and the resulting structure moves away from the ideal hexagonal arrangement.

Similarly, the deposition can be influenced by selective modification of the substrate, for example by roughening, hydrophobising/hydrophilising, functionalising or the like.

Further subject matter of the present invention are biomarker detection sensor kits (sensor kits) comprising or consisting of
A) at least one biomarker detection sensor as set forth above,
BO) a preparation comprising at least one biomarker-specific aptamer having a fluorophore bound thereto,
C) optionally further analysis material, preferably cuvettes, pipettes, light source(s), fluorescence detector, in particular fluorescence spectrometer.

Still further subject matter of the present invention are biomarker detection sensor kits (sensor kits) comprising or consisting of
A) at least one biomarker detection sensor as set forth above,
BO) a preparation comprising at least one biomarker-specific aptamer having a fluorophore bound thereto,
   optionally
B1) at least one further preparation comprising at least one identical biomarker-specific aptamer with a different fluorophore bound thereto, and/or
B2) at least one further preparation comprising at least one other biomarker-specific aptamer having another fluorophore bound thereto,
C) optionally further analysis material, preferably cuvettes, pipettes, light source(s), fluorescence detector, in particular fluorescence spectrometer.

In variant B2), it can be distinguished between two preferred embodiments in B2i) at least one, preferably exactly one, other biomarker-specific aptamer for the detection of the same biomarker but binding to a different epitope of the biomarker with a different fluorophore bound thereto, and

B2ii) at least one, preferably exactly one, other biomarker-specific aptamer for the detection of a further biomarker with a different fluorophore bound thereto, and accordingly be selected from B2i) and/or B2ii).

The advantages of B2) are that B2i), by binding two aptamers to the same biomarker, confirms the detection of that biomarker and is thus even more reliable than B1) and that B2ii) can detect a second biomarker.

“Same” and “different” in B1), B2) respectively refer to BO).

Where a single variant is described/discussed below, this is BO) or any one, unless otherwise stated.

In connection with the term “kit”, it should be noted that this kit does not necessarily have to be suitable for the “trouser pocket”. Rather, it is intended to express that prior to analysis, both the biomarker detection sensor as described above as well as the preparations comprising biomarker-specific aptamer(s) are present separately, although they are matched and associated with each other. In addition, the kit is also intended to be able to comprise several detection sensors and several aptamer preparations, which are also designed for different biomarkers.

In this context, it is also readily apparent to the person skilled in the art that the further analysis material is not necessarily limited to the cuvettes, pipettes, fluorescent light source and fluorescent detector just mentioned, but may comprise further material within the scope of the usual.

In embodiments of the present invention, it is possible that the biomarker detection sensor kit is configured as a transportable kit. In this context, a transportable light source is then designed, for example, in the form of a torch and the fluorescence detector is accordingly also designed in a comparatively handy form. For this purpose, it may be sufficient in variants if the fluorescence detector merely displays a fluorescence signal, but cannot necessarily represent its intensity quantitatively. This would be an example of a rapid test kit. However, it is equally possible that in the context of the present invention the biomarker detection sensor kit is designed on a laboratory scale, i.e. in particular both the fluorescent light source as well as the fluorescent detector are suitable and designed for permanent laboratory operation.

Within the scope of the present invention, it is possible that the biomarker-specific aptamer is in principle suitably selected for any disease-specific biomarker.

However, it is essential that the biomarker-specific aptamer is concordantly targeted to the same biomarker in accordance with the anti-biomarker receptor bound to the gold nanoparticles, whereby aptamer and antibody are targeted to different epitopes of the biomarker (in the event that aptamer and antibody were targeted to the same epitope, they would compete with each other and worsen the result).

In the context of the present invention, it is preferred if the biomarker-specific aptamer is a malaria-specific aptamer.

In particular, it is preferred in the context of the present invention that the biomarker-specific aptamer is the malaria 2008 aptamer.

Furthermore, in principle any fluorophore that can be chemically bound to this biomarker is suitable. Examples of useable fluorophores are 5-FAM (5-carboxyfluorescein), cyanine 7, cyanine 5, cyanine 3b or quantum dots.

In practice, 5-FAM and cyanine 5 have well stood the test as fluorophores for such or similar investigations.

Accordingly, it is preferred in the context of the present invention if 5-FAM and/or cyanine 5 are bound to the biomarker-specific aptamer as fluorophore.

Thus, the most preferred biomarker-specific aptamer in the context of the present invention is the malaria 2008 aptamer with cyanine 5 attached thereto, provided that a single fluorophore is used.

In further preferred embodiments of the present invention, it is preferred if two different fluorophores are used at the same time.

This then leads to an overlap of the plasmon resonances and can thus be used for a further amplification of the signal.

In principle, all fluorophores that can be chemically bound to the biomarkers are also suitable in these embodiments.

It is particularly preferred if 5-FAM and Cy5 as fluorophores are combined. In this case, the plasmon resonance is designed in such a way that it is coupled to the emission peak of 5-FAM with both the excitation peak and the emission peak of Cy5, thus achieving, by emission rate enhancement, a 160-fold fluorescence enhancement and, by a dual mechanism (excitation and emission rates), a 5200-fold signal enhancement.

The combination of both fluorescence detections extends the detection concertation range by two orders of magnitude compared to detection of only a single fluorophore in complex matrices, such as human blood. Furthermore, the confirmation of the analyte binding by two redundant fluorescence signals improves the reliability of the signal and reduces falsely positive signals due to non-specific binding. Alternatively, the proposed approach can be used for the simultaneous monitoring of different biomarkers at low concentrations, paving the way for potential multiplex and high-throughput analysis.

In these embodiments, two different biomarker-specific aptamers can be used, or the same.

Thus, in these embodiments, different biomarker-specific aptamers with the same fluorophores or different fluorophores can be used to detect different biomarkers simultaneously, or the same biomarker-specific aptamer with different fluorophores can be used to detect the same biomarker but at different wavelengths.

Analogous procedures and embodiments with three or even more different fluorophores and/or biomarker-specific aptamers are also encompassed by the present invention.

Thus, many different variations can be covered by the present invention, allowing a high degree of flexibility, especially when used in the form of kits.

It is noteworthy that with the variant of the present invention with two different fluorophores, in particular 5-FAM and Cy5, the accessible detection range by using both fluorophores is extended, with Cy5 being more sensitive in the lower concentration range (up to 0.1 pM), while 5-FAM covers large concentrations (up to 1 μM). In this way, for example, it is possible to measure over seven orders of magnitude instead of four or five for one fluorophore alone.

In this respect, it has also been part of the present invention to enable multiplex detection—while maintaining high quality performance—and a correspondingly developed dual-resonant plasmonic nanostructure which is suitable for simultaneous detection of two different analytes in the matrix of interest is also part of the present invention.

To this end, in one embodiment of the present invention, branching patterns of plasmon-coupled honeycomb AuNPs that generate a collective mode whose resonance is in the far red region and interspersed plasmon-decoupled AuNPs that exhibit a narrow localised surface plasmon resonance (LSPR) at 524 nm are prepared by block copolymer micelle nanolithography (BCMN).

Said preparation comprising at least one biomarker-specific aptamer with fluorophore bound thereto may be said aptamer per se, but may also comprise other substances in addition to the aptamer with fluorophore bound thereto, as are known in the art for the use of such aptamers with fluorophores bound thereto. For example, the fluorophore-labelled aptamer may be present in aqueous solutions, wherein these aqueous solutions may, for example, still be buffered or may also contain preservatives or the like. In embodiments of the present invention, for example, 10 millimolar phosphate-buffered salt solutions (PBS) are well suited.

This applies mutatis mutandis to all variants BO), B1) and B2).

It is also essential for these biomarker detection sensor kits that the biomarker detection sensors are designed as described above.

This is because the combinations of the biomarker detection sensors as described above with the preparations comprising at least one biomarker-specific aptamer enable a high selectivity.

In particular, the selectivity for the P<DH preferred to be detected according to the invention is achieved by the combination of the anti-pLDH antibody bound to the gold nanoparticles via thiol groups and the malaria 2008 aptamer with cyanine 5 bound thereto as a fluorophore.

In the context of the present invention, it was found that for the embodiment with a fluorophore at a diameter D of the nanoparticles of 48 or 50 nm and an interparticle distance (edge-to-edge) din the two-dimensional lattice of 19 or 20 nm between the individual gold nanoparticles having anti-pLDH antibodies bound thereto via thiol groups, a particularly strong fluorescence enhancement could be achieved in combination with the malaria-2008 aptamer with fluorophore cyanine 5 bound thereto.

Accordingly, a particularly high specificity and sensitivity can be achieved with this particularly preferred embodiment.

The fluorescence intensity depends on the concentration of the biomarker. The intensity increases with increasing concentration of the biomarker and asymptotically approaches a limit value. This limit value is reached when all antibodies are occupied by biomarkers bound to them.

Until all antibodies are fully occupied, a quantitative determination of the biomarker concentration is possible by determining the fluorescence intensity.

From the time when all antibodies are fully occupied with biomarkers, there is no longer any quantification, but only a qualitative indication (as before, of course, in addition).

In embodiments of the present invention, biomarker concentrations between 0.001 femtomole and 100 nanomoles, in particular between 0.01 femtomoles and 10 nanomoles, can be quantitatively determined. Higher concentrations can be determined qualitatively.

Qualitative determination means here that the presence of the biomarker is indicated by fluorescence, but the exact amount is not determined or, in the case of complete occupancy of all antibodies, cannot be determined.

For the use of the present invention as a rapid test or point-of-care analysis, the qualitative determination is usually sufficient. The quantitative determination is usually used for more precise clinical investigations.

It is a great advantage of the present invention that both are possible.

In this context, it should be noted that for other fluorophores, other dimensions may be more advantageous in terms of diameter D and distance d. This can be precisely coordinated in individual cases.

At the same time, it should be noted that the order symmetry of the two-dimensional lattice of the gold nanoparticles can have an influence on the plasmon resonance. In the context of the present invention, a particularly good plasmon resonance has been achieved with hexagonal or hexagonal close-packed gold nanoparticles; however, cubic close-packed, random close-packed or even other, order symmetries are also encompassed by the present invention.

For the particularly preferred embodiments of the present invention with a hexagonal or hexagonal close packing of the gold nanoparticles, anti-pLDH antibodies and malaria 2008 aptamer with cyanine 5 bound thereto, for D=40 to 60 nm, preferably 44 to 55 nm, particularly preferably 46 to 52 nm, especially preferably 48 or 50 nm and d=16 to 24 nm, preferably 17 to 22 nm, particularly preferably 18 to 21 nm, especially preferably 19 or 20 nm, a particularly high enhancement of the fluorescence was observed and found in the context of the present invention, since the generated plasmon resonance overlaps with the fluorophore absorption and fluorophore emission; this is also referred to as dual amplification in the context of the present invention, because both processes are amplified simultaneously.

In this context, it should also be pointed out that the dual amplification for the variant of hexagonal arrangements and with a biomarker-specific aptamer and a fluorophore bound thereto by the plasmon resonance reaches its maximum at about 10 nm distance from the particle surface. By the combination of antibody/P<DH/aptamer just described precisely this distance in the context of this variant of the present invention is achieved.

Accordingly, by this particularly preferred embodiment of the present invention a particularly strong dual amplification and particularly high selectivity and efficiency are achieved.

This is a major advantage of the present invention over a sandwich assay as known in the prior art, since two antibodies are used in such sandwich assays. However, these two antibodies would lead to the distance between the fluorophore and the particle surface becoming too large and the corresponding amplification becoming smaller. This would then lead to poorer detection limits and lower efficiency.

In this respect, the present invention is greatly improved with respect to sandwich assays known in the prior art.

Furthermore, a method for determining disease-specific biomarkers in body fluids is a subject matter of the present invention. This method comprises or consists of the following steps:
Step i) consists of the provision of a biomarker detection sensor as described above.

As step ii), a body fluid sample is then applied to the sensor and mixed with at least one biomarker-specific aptamer having a fluorophore coupled thereto.

This can happen in such a way that the body fluid sample and the at least one biomarker-specific aptamer having a fluorophore coupled thereto are applied to the sensor simultaneously, or that the at least one biomarker-specific aptamer having a fluorophore coupled thereto is added before or after. Similarly, it is possible to directly apply a pre-prepared mixture of body fluid sample and at least one biomarker-specific aptamer having a fluorophore coupled thereto to the sensor.

The sample applied to the sensor is then irradiated by means of a light source in step iii). The light source is selected in such a way that the fluorophore used is particularly well excited. In preferred embodiments, this light source is a light source that emits in the red region of the visible spectrum; particularly advantageous results are obtained if the light source emits in a wavelength range between 610 and 670 nm, preferably between 625 and 655 nm, in particular, in the case of cyanine 5 as fluorophore, at 625 nm. For the case of 5-FAM as fluorophore, particularly preferred results are obtained when the light source emits wavelengths from 450 nm to 510 nm, in particular 470 nm.

Next, as step iv), the fluorescence emission of the sample is detected.

In principle, this detection can be carried out using all methods known in the art. Provided that the method according to the invention is carried out with one of the biomarker detection sensor kits according to the invention, this can also be a purely visual detection by the person carrying out the method, in which case the detection would simply be carried out via the optical impression obtained by the person carrying out the method.

Preferably, however, the detection is performed with dedicated fluorescence spectrometers, for example those based on CMOS or sCMOS photodetectors. For example, for excitations by a light source with wavelengths from 625 to 655 nm, emissions from 665 to 715 nm are obtained for the anti-pLDH antibody/P<DH/Malaria2008 aptamer-Cy5 case, and emissions from 500 to 550 nm are obtained for an excitation with wavelengths from 450 to 490 nm for the anti-pLDH antibody/P<DH/Malaria2008 aptamer-5FAM case.

The at least one biomarker-specific aptamer having a fluorophore coupled thereto to be mixed with the body fluid sample is as described above and may also be in the form of the preparation described for the biomarker detection sensor kit containing said biomarker-specific aptamer having a fluorophore coupled thereto.

As an optional step v), analysis of fluorescence emission may be performed in the method according to the invention. This analysis may be quantitative as well as qualitative or both. Suitably, this analysis is performed using commercially available computers and fluorescence data processing programs known to those skilled in the art.

Further optionally as step vi) in the method of the present invention, the determined result of the fluorescence emission analysis may be stored or displayed or both.

Although in the method for determining disease-specific biomarkers according to the present invention, the body fluid is not limited to a specific body fluid, it is natural for the person skilled in the art to select the body fluid based on whether the disease-specific biomarkers he is looking for are present in the particular body fluid.

In preferred embodiments of the present invention, the body fluid is a blood sample.

The body fluids may be processed or prepared prior to use in the method of the present invention in accordance with procedures commonly used in the art.

It should be noted that the method of the present invention is particularly suitable and intended for examining body fluid samples that are already available as such, i.e. the collection of body fluids is not part of the method of the present invention.

It is preferred in the method of the present invention that the anti-biomarker receptors are anti-pLDH antibodies, and the biomarker-specific aptamers are malaria-2008 aptamers, to which 5-FAM and/or cyanine 5 is/are preferably bound as a fluorophore; in accordance with the above discussion of the biomarker detection sensor or biomarker detection sensor kit of the present invention.

By the pre-treatment of the anti-pLDH antibodies with UV radiation these are activated. As part of this activation, the antibodies are converted into a conformation such that they bind to the gold nanoparticles in a manner that makes the binding epitopes of the antibody readily accessible to the biomarker to be detected.

Subject matter of the present invention are also uses of the biomarker detection sensors, of the biomarker detection sensor kits or of the method for the determination of disease-specific biomarkers in body fluids according to the invention for the qualitative or quantitative determination of disease-specific biomarkers.

In particular, this use is encompassed for the determination of malaria biomarkers and in particular in body fluid samples, most preferably in blood samples.

Another preferred use according to the present invention is the use of the biomarker detection sensors according to the present invention as substrate in automated multititer plates.

The biomarker detection sensors according to the present invention can be analogous to Lohmüller, T., Aydin, D., Schwieder, M. et al, “Nanopatterning by block copolymer micelle nanolithography and bioinspired applications”, Biointerphases 6, MR1-MR12 (2011), https://doi.org/10.1116/1.3536839.

In the following, the invention will be explained in more detail with reference to the figures. The figures are not necessarily to scale and are simplified. For example, common measures etc. familiar to those skilled in the art are not necessarily shown (screws, valves, reaction vessels, exact molecular structure etc.) in order to facilitate the readability of the figures.

### FIGURE DESCRIPTION

FIG. 1 is a schematic representation of the procedure for preparing a biomarker detection sensor according to the present invention.

The preparation of these arrays of gold nanoparticles was initially carried out via nanolithography based on the self-assembly of block copolymers, as illustrated in section A). For this purpose, block copolymers 7 are used, which have a hydrophilic chain (shown by dash) and a hydrophobic chain (shown by block). By mixing with a non-polar solvent, in particular toluene T, inverse micelles 8 are formed, i.e. micelles in which the core is hydrophilic and the outer shell is hydrophobic. A gold salt Au-S (for example HAuCl₄) is then added to this mixture containing the inverse micelles, causing the inverse micelles to take up gold precursors 9 in their centre, the hydrophilic core. These micelles can then be deposited on a substrate 1, for example by means of dip coaters. Due to their structure, the micelles then arrange themselves in regular structures on the surface of the substrate. Afterwards, the micelles around the gold precursors are removed, for example by means of plasma furnace treatment. By this, the gold precursors 9 are also reduced to the gold nanoparticles 2. A two-dimensional lattice of gold nanoparticles 2 (unfilled circles) then remains on the surface.

In Section B) it is illustrated how a solution of anti-pLDH antibodies 10 is pre-treated by UV radiation (shown as a flash) and then subsequently this treated aqueous solution 10a is poured onto the substrate with gold nanoparticles thereon prepared in section A), whereby the antibodies bind to the gold nanoparticles.

As a result, the substrate 1 illustrated in section C) is obtained with gold nanoparticles 2 thereon to which the anti-biomarker receptors are bound, which is shown here by filled circles. This fabrication method is also described, for example, in Lohmueller, T., Aydin, D., Schwieder, M. et al, “Nanopatterning by block copolymer micelle nanolithography and bioinspired applications”, Biointerphases 6, MR1-MR12 (2011), https://doi.org/10.1116/1.3536839.

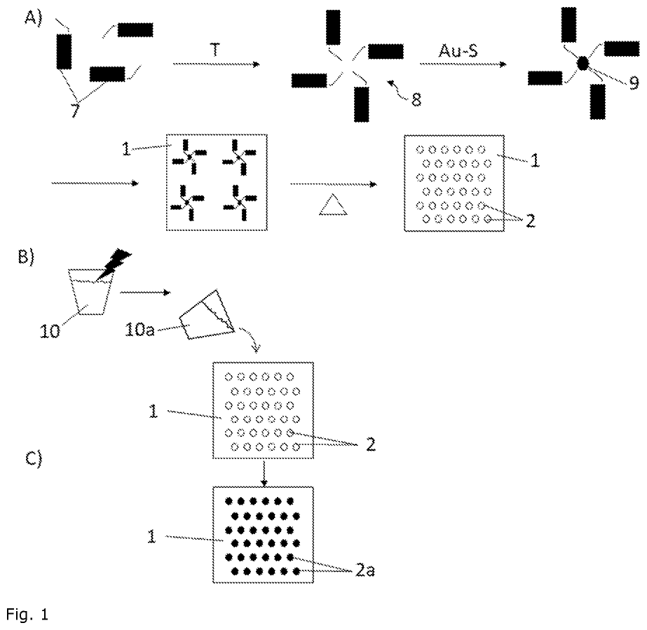

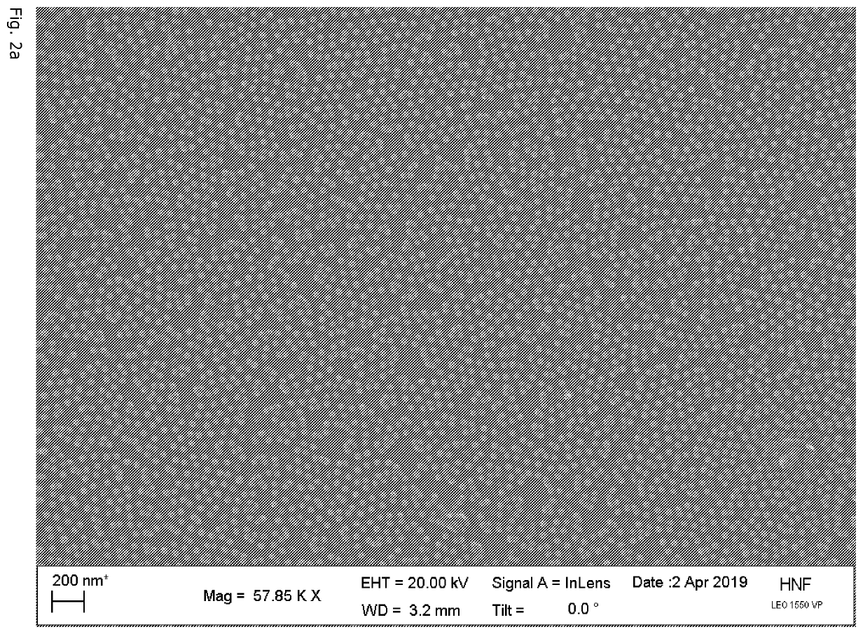

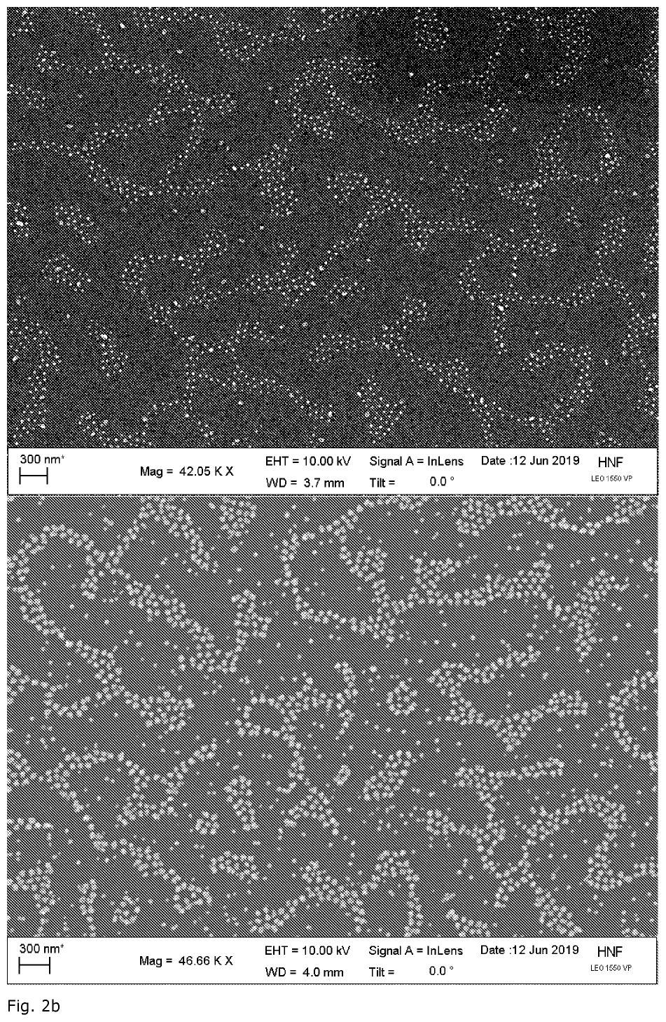

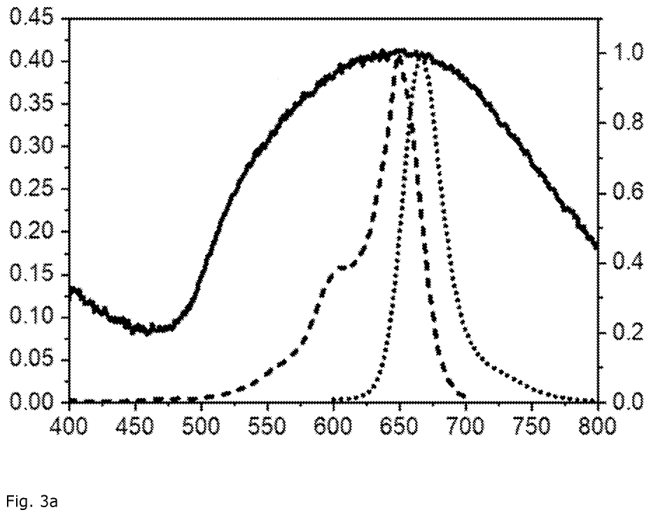

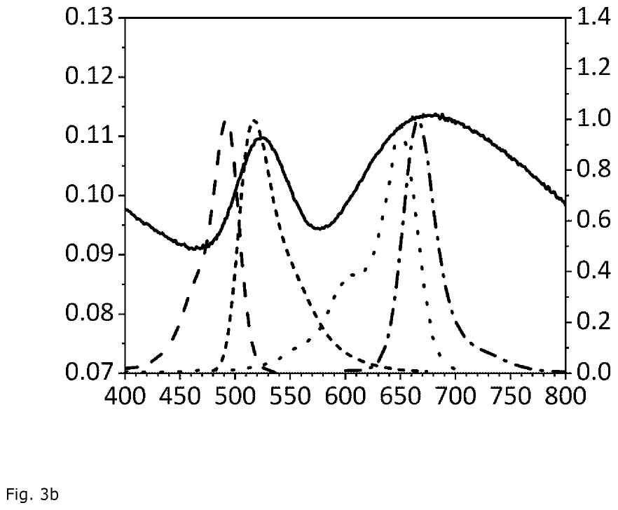

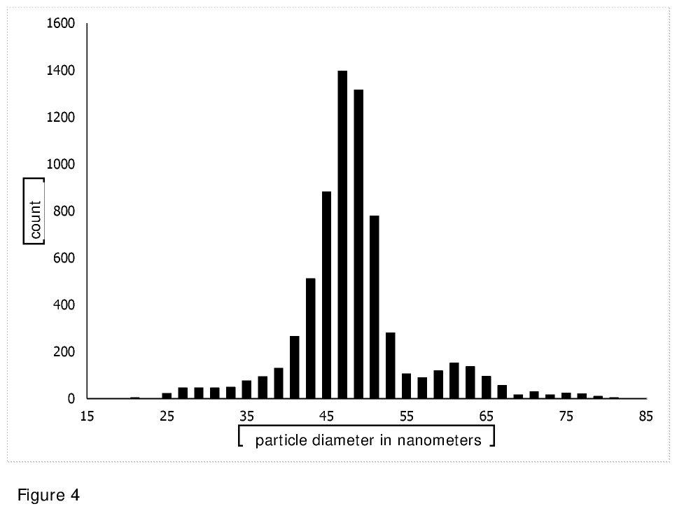

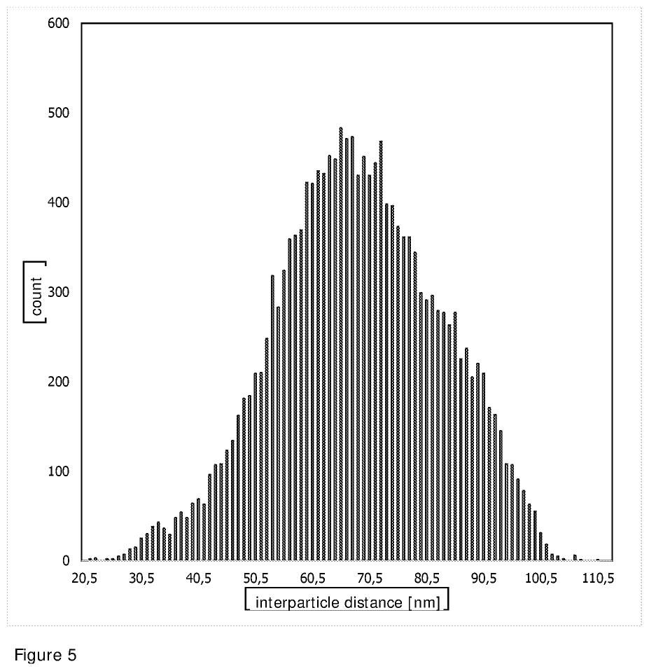

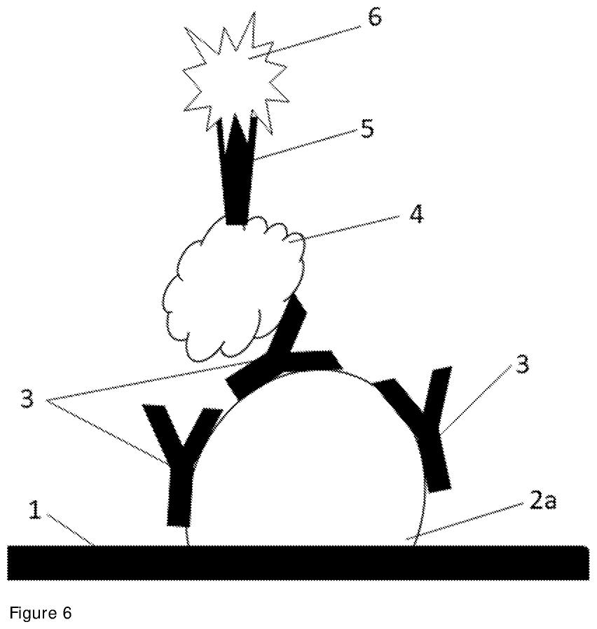

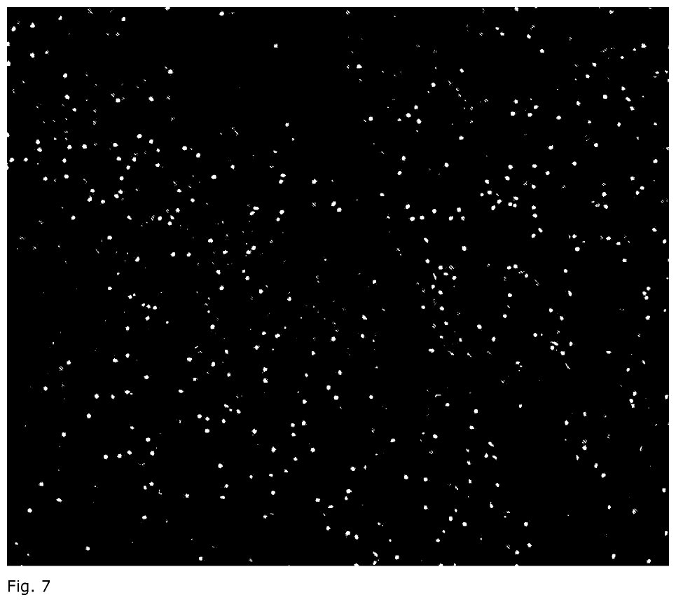

FIG. 1 illustrates the arrangement of the particles on the substrate only schematically and is not limited to any particular arrangement. The production method illustrated in this figure can be used for the production of substrates with nanoparticles arranged hexagonally thereon (as illustrated), as well as for the production of branched nanoparticles or also for mixed forms.

FIG. 2a is a scanning electron microscope image of a substrate provided with ordered gold nanoparticles according to a variant of the present invention. From this image it can be seen that defects can certainly occur during the manufacture of the sensors. However, these defects are not detrimental to the overall performance of the biomarker detection sensor, as they are averaged out, so to speak, in the context of the plasmon resonance or dual amplification.

FIG. 2b are two scanning electron micrographs of another substrate provided with ordered gold nanoparticles according to another variant of the present invention. From these images it can be seen that in this case hexagonal structures are still present in the near order, but the far order shows essentially branched structures as well as isolated nanoparticles. The upper image shows a structure before and the lower image one after the AuNP growth.

FIG. 3 is a diagram in which the two narrower curves represent the excitation spectrum (left, dashed curve) and the emission spectrum (right, dotted curve) of cyanine 5 (Cy5) and the broad curve represents the experimentally determined extinction spectrum of a malaria biomarker detection sensor (prepared according to example A) described below). It can be seen that the broad extinction spectrum allows the overlapping of the plasmonic resonance over both extinction as well as emission spectra of cyanine 5. The wavelength in nm is plotted on the x-axis and the extinction (left) and normalised excitation/emission (right) on the y-axis.

FIG. 3b for a substrate prepared according to example B) described below shows the experimental (solid line) extinction spectrum. In addition, the plasmon fluorophore overlap with 5-FAM (emission spectrum, small dashed line//excitation spectrum, large dashed line) and Cy5 (emission spectrum, dash-dot line//excitation spectrum, dotted line) dyes is shown. It can be seen that the experimental extinction spectrum shows two plasmonic resonances at 524 nm and 675 nm. Isolated AuNPs contribute to the resonance at lower wavelength, as expected from 30 nm gold nanospheres in air, whereas AuNPs arranged along the branches lead to a collective resonance at higher wavelength. The extinction is plotted on the left axis, excitation/emission on the right axis. The wavelength in nm is plotted on the horizontal axis.

FIG. 4 is a diagrammatic representation of the particle size distribution of gold nanoparticles prepared according to example A) described below. The mean diameter D of the gold nanoparticles in this example is about 48 nm (47.67±0.14 nm).

FIG. 5 is a diagrammatic representation of the interparticle distances in nanometres relative to the respective centres of the nanometres. The average interparticle distance, measured centre to centre, is about 68 nm (68.47±0.19 nm)), resulting in an interparticle distance, measured particle edge to particle edge, of about 20 nm (produced according to example A) described below).

FIG. 6 schematically illustrates the mode of operation of the present invention by way of a single gold nanoparticle (AuNP). The basis is a gold nanoparticle 2 arranged on a substrate 1, to which antibodies 3 (shown here as Y) are bound. In this figure, it becomes clear from the way the antibodies 3 are shown that they are bound to the gold nanoparticles 2 in a conformation that makes the binding epitopes of the antibodies readily accessible to the respective biomarker. This is illustrated in that one “arm” of each antibody 3 is oriented away from the gold nanoparticle 2. Furthermore, the biomarker 4 (P<DH) under investigation is shown (only one, for clarity, in the form of a cloud), which is shown here as binding to the binding epitope of the middle antibody 3. On the other side of the biomarker 4, an aptamer 5 is then shown, which thus, so to speak, grips the biomarker 4 together with the antibody 3 (sandwich). On the side of the aptamer 5 facing away from the biomarker 4, the fluorophore 6 is shown as a star which is intended to represent its fluorescence emission. The distance between the fluorophore 6 and the surface 1 is here about 10 nm, whereby with this arrangement both a high sensitivity as well as a high selectivity are achieved.

FIG. 7 shows an image of the fluorescence dots of a sensor assembly with detected biomarker according to example B), below. The image shows a reproduction in which the red and green channels resulting from detection with two colour channels, i.e. two different fluorophores (5-FAM and Cy5), are shown united (here in black and white for reproduction purposes). One can clearly see the fluorescence dots and thus the good detectability that can be achieved with the present invention. A corresponding detectability also applies to the variant according to example A) (not shown).

### LIST OF REFERENCE SIGNS

In the figures, same reference signs mean same materials, substances, etc.
1 substrate
2 gold nanoparticle
2a gold nanoparticles with bound antibodies
3 antibody
4 biomarker
5 aptamer
6 fluorophore
7 block copolymer
8 invers micelle
9 gold nanoparticle precursor
10 solution of anti-pLDH antibodies (untreated)
10a solution of anti-pLDH antibodies (after UV treatment)
T toluene
Au-S gold salt, in particular HAuCl₄

The present invention will now be explained in more detail with reference to the following non-limiting examples. The following non-limiting examples serve to illustrate the embodiments embodied therein. It is known to person skilled in the art that variations of these examples are possible within the scope of the present invention.

### EXAMPLES

### 0. Materials and Chemicals

Diblock copolymers (P18226-S2VP and P3807-S2VP, respectively) were purchased from Polymer Source Inc (Dorval, Canada) and were prepared from polystyrene(x)-b-2-polyvinylpyridine(y) (PS(x)-b-P2VP(y)), wherein x=30000 and y=8500, or x=325000 and y=92000 indicate the respective molecular weight of polystyrene (PS) and poly(2-vinylpyridine) (P2VP) (The number average (M_{N}) of the distribution is indicated in each case. The ratio of M_{w}:M_{N} for P18226-S2VP is 1.06 and indicates a very monodisperse distribution). Toluene (99.8%), gold(III) chloride trihydrate (HAuCl₄*3H₂O), silver nitrate (AgNO₃) and ascorbic acid were purchased from Sigma-Aldrich; acetone (≥99.0%), 2-propanol (≥99.5%) and ethanol (≥99.5%) were purchased from Merck Millipore; Hexadecyltrimethylammonium bromide (CTAB) (≥99.0%) was purchased from Fluka; bovine serum albumin (BSA) (fraction V IgG-free, low in fatty acids) came from Gibco. High purity deionised water used for all aqueous solutions was dosed from a Milli-Q® system (18.2 megaohm specific resistance).

10 mM phosphate buffered saline (PBS) (NaCl 10 mM, NaH2PO4 10 mM, Na₂HPO₄ 10 mM, MgCl₂ 1 mM, pH 7.1) and 25 mM Tris-HCI buffer (NaCl 100 mM, imidazole 20 mM, Tris (=Tris(hydroxymethyl)aminomethane) 25 mM, HCI 25 mM, pH 7.5) were prepared by dissolving the reagents (purchased from Sigma-Aldrich) in high purity water. Pan-malaria antibody (anti-pLDH monoclonal antibody clone 19 g7) was prepared by Vista Laboratory Services (Langley, USA). Recombinant Plasmodium falciparum lactate dehydrogenase (P<DH) and P<DH were obtained by bacterial expression. Malaria 2008s aptamers labelled with 5-FAM or cyanine 5-tag (5′-5-FAM(Cy5)-CTG GGC GGT AGA ACC ATA GTG ACC CAG CCG TCT AC-3′) were produced by Friz Biochem GmbH (Neuried, Germany). Millex® syringe filters (pore size 0.20 μm) with hydrophobic polytetrafluoroethylene membrane were purchased from Merck Millipore; Superslip® coverslips (borosilicate glass, thickness 0.13-0.17 mm) were purchased from Thermo Fisher Scientific and cut by a diamond-tipped glass cutter.

### EXAMPLE A

1. fabrication of an ordered array of AuNPs for the detection of PfLDH in blood

Nanolithography based on self-assembly of block copolymers was used to prepare arrays of ordered AuNPs with adjustable density, size and interparticle distance. 29.2 mg of diblock copolymers P18226-S2VP were added to 15 ml of toluene under vigorous stirring and controlled conditions (argon inert gas, O₂<1 ppm, H₂O<0.1 ppm) and kept for 72 h to obtain a homogeneously dispersed invers micelles with hydrophilic core and outer hydrophobic shell (spherical). Then, 15.7 mg HAuCl₄*3H₂O were added to the solution for 72 hours to incorporate the gold precursor into the hydrophilic core of the micelles, resulting in the formation of AuNPs encased in a hydrophobic shell. Once the gold powder was fully dispersed, the yellowish solution was filtered to remove micelle aggregates and impurities. While maintaining agitation, this solution could be stored for at least six months.

Before the PS-AuNPs were deposited on the glass coverslips (which had a size of 10 ×8 mm²), the substrates were cleaned by ultrasound for five minutes successively in acetone, 2-propanol and pure ethanol and dipped in toluene to “make” the surface non-polar so that the hydrophobic shells could adhere to it. Subsequently, the substrates were immersed in the solution containing PS-AuNPs using an immersion coater with careful adjustment of the immersion speed. It was found that an immersion speed of 0.6 mm/s enabled a particularly good coating of the glass surface, both in terms of the density of the arrangement of the AuNPs as well as in terms of the large-area uniformity of the deposition. The PS-AuNPs were transferred to the non-polar glass surface by hydrophobic interaction, resulting in a hexagonal arrangement by self-assembly. A corresponding process is also illustrated in FIG. 1. Afterwards, the copolymers were etched by oxygen plasma treatment (0.8 mbar pressure, 200 W, 30 minutes), immobilising the AuNPs at prefixed positions on the glass surface.

Because the plasmonic resonance strongly depends on the ratio R between the AuNP diameter D and the interparticle distance d, higher values of R guarantee a larger coupling between the AuNPs. For R>2/3, the plasmonic resonance is dominated by the collective behaviour of the AuNPs, and several advantages for metal-enhanced fluorescence (MEF) result. To enhance R, the substrates were incubated with 2 ml of an Au deposition solution (CTAB 190 mM, HAuCl₄*3H₂O 42 mM, AgNO₃ 8 mM, ascorbic acid 100 mM) for 2 h in the absence of light. Afterwards, the substrates were rinsed abundantly with high purity water and stored in the absence of light until use. The characterisation of the nanostructured substrates was done by UV-VIS spectroscopy and scanning electron microscopy (SEM).

The results of the measurements are shown in FIGS. 2 to 5.

2. functionalisation

The functionalisation of the AuNPs with pan-malaria antibodies was performed by photochemical immobilisation technique (PIT). For this purpose, 1 ml aqueous solution of anti-pLDH (25 μg/ml) were irradiated by a UV lamp for 30 seconds and then poured onto the substrate. The UV source consisted of two U-shaped low-pressure mercury lamps (2 W at 254 nm) into which a standard quartz cuvette could be inserted. Taking into account the geometry of the lamps and the proximity to the cuvette, the radiation intensity used to generate the thiol groups was about 1 W/cm ². This intensity was so weak that a direct photolysis of the disulphide bridges, which absorb only weakly at 254 nm, is avoided. Only the disulphide bridge of the Cys-Cys-Trp triad (where the tryptophan “activates” the disulphide bridge) was cleaved by this. Functionalisation via PIT ensured that the antibodies (Abs) were covalently linked to the gold surface and their binding sites were accessible to the environment.

3. washing

The samples were rinsed with high purity water (dosed using a Milli-Q® system) to remove unbound antibodies.

4. blocking

1 ml bovine serum albumin (BSA) solution (50 μg/ml) was applied to cover the free gold surface and protect it from non-specific adsorption.

5. washing

The samples were rinsed copiously with high purity water. Storage until use was in PBS solution (10 mM) at room temperature.

6. analyte detection (P<DH fixation by immobilised Abs)

The desired amount of Plasmodium falciparum lactate dehydrogenase (P<DH) was added to 1 ml of a diluted solution of uninfected human blood (dilution 1:100 in 25 mM Tris buffer). The functionalised substrates were incubated with 1 ml of contaminated blood solution of the same dilution for 2 hours at room temperature. A rocking shaker was used to accelerate binding kinetics and improve analyte diffusion. Concentrations between 0.01 femtomoles and 10 nanomoles were measured.

7 washing

The samples were rinsed copiously with Tris buffer (25 mM) and with high purity water to remove unbound proteins.

8. fluorescence aptamer-based assay

The samples were transferred to 1 ml of 10 mM phosphate-buffered saline (PBS), wherein here the PBS still contains 0.1 μM malaria 2008s aptamers labelled with Cy5 tag (5′-Cy5-CTG GGC GGT AGA ACC ATA GTG ACC CAG CCG TCT AC-3′) (i.e. the malaria2008 aptamer with fluorophore bound to it. The solution was gently shaken in the absence of light for 2 hours using a rocking shaker. The result was a sandwich arrangement. Such an arrangement is exemplary also shown in FIG. 6.

9 washing

The samples were rinsed abundantly with PBS and high purity water to remove unbound aptamers.

10. measurement of the fluorescence signal

Fluorescence images were using a Zeiss Axio Observer Z1 phase-inverted contrast fluorescence microscope equipped with Zeiss Colibri.2 LED light source (module 625 nm), Zeiss Plan-Apochromat lens 10×/0.45 Ph 1 M27 (FWD=2.1 mm), Kubus 50 Cy5 filter (excitation 625-655 nm/emission 665-715 nm) and pco.edge 5.5 sCMOS photodetector (scale 0.650 μm×0.650 μm per pixel, image size 2560×2160 pixels, scaled image size 1.66 mm×1.40 mm, 16 bit dynamic range, 2 seconds exposure time for each image). The taken fluorescence images were processed with ImageJ software. The “rolling ball” algorithm was used to calculatively remove the difference in brightness between the centre and the edge of the image caused by the optics. The background was measured locally for each pixel by average forming over a circle around the pixel. Such a value is then subtracted from the original image, smoothing out spatial variations of the background. The “rolling ball” radius was set to 10 pixels, a size sufficiently larger than the size of the largest objects that were not part of the background. A threshold slightly higher than the smoothed background was set to segment the image, the overall intensity of which was measured by summing the signal components from all spots. To obtain a good and reliable analysis of the fluorescence signals, ten images were taken randomly from each sample and their intensity average was determined.

11. simulation of the E-field around the gold nanoparticles

The optical response of the 2D AuNP frameworks was simulated by the “FDTD Solutions” tool implemented in Lumerical software. A linearly polarised electromagnetic radiation that ran along the z-direction was used to study the system. Virtual (simulated) photo-detectors (PDs), sensibly placed in the working space, could measure the intensity of the electromagnetic field as a function of time. A photo detector was assigned to measure the excitation spectrum of the nanostructure. Symmetric/antisymmetric boundary conditions (BCs boundary conditions), set along the x and y directions, extend the plasmonic excitation over an infinite 2D framework and at the same time reduce the simulation time by a factor of 8 without degrading the accuracy of the results. Bloch BCs (periodic boundary conditions) were used only for polarisation studies to compensate for the phase shift that occurs when an electromagnetic disturbance when a non-zero angle reintroduced on the opposite side of the working space. Perfectly adjusted layer BCs in the z-direction ensure perfect absorption of the electromagnetic waves backscattered by the plane containing the light source and incident through the opposite side of the working environment. The working environment was resolved over a grid with a spatial resolution of 0.5 nm, ensuring high accuracy while keeping the simulation time within a few hours. The AuNPs were modelled as homogeneous gold hemispheres, whereas the substrate was represented as a thick dielectric layer of silicon dioxide (SiO₂).

### EXAMPLE B

Essentially corresponding to Example A) above, the following procedure was followed:

1. preparation of an array of AuNPs

24.3 mg of the diblock copolymers P3807-S2VP were added to 15 ml of toluene with vigorous stirring and kept for 72 hours to obtain a homogeneous, monodisperse solution of invers micelles. Then 13.1 mg HAuCl₄*3H₂O were added to the solution for 72 hours to allow the formation of gold nuclei encased in polystyrene shells (PS-AuNPs). The yellowish solution was then filtered to remove possible copolymer aggregates. Diblock copolymers and gold(III) chloride trihydrate were handled in a glovebox under inert gas (argon) and controlled conditions (O₂<1 ppm, H₂O<0.1 ppm).

Before the PS-AuNPs were deposited on the glass coverslips (10×8 mm²), the substrates were cleaned by ultrasound for five minutes successively in acetone, 2-propanol and ethanol and then immersed in a non-polar solvent to allow the adhesion of hydrophobic polystyrene shells. Subsequently, the substrates were immersed in the solution containing PS-AuNPs using an immersion coater and an immersion speed of 0.8 mm/s. This immersion speed allows a good coating of the substrate surface while preventing maximum packing density (cf. FIG. 2b, from which it can be seen that in the near order hexagonal structures are still given, but the far order shows branched structures). Afterwards, the copolymers were etched by oxygen plasma treatment (0.8 mbar pressure, 200 W, 30 minutes), which immobilised the AuNPs at prefixed positions on the glass surface. Subsequently, the substrates were incubated with 2 ml of an Au deposition solution (CTAB 190 mM, HAuCl₄*3H₂O 42 mM, AgNO₃ 8 mM, ascorbic acid 100 mM) for 2 hours in the absence of light.

2. functionalisation

The functionalisation of the AuNPs with pan-malaria antibodies was performed by photochemical immobilisation technique (PIT). For this purpose, 1 ml aqueous solution of anti-pLDH (50 μg/ml) was irradiated by a UV lamp for 30 seconds and then poured onto the substrate. The UV source (Trylight, Promete S.r.l) consisted of two U-shaped low-pressure mercury lamps (6 W at 254 nm) into which a 10 mm standard quartz cuvette could be inserted. Taking into account the geometry of the lamps and the proximity to the cuvette, the radiation intensity used to generate the thiol groups was approximately 0.3 W/cm².

3. washing as above
4. blocking as above
5. washing as above
6. analyte detection (P<DH fixation by immobilised Abs)

Uninfected human blood was diluted 1:100 in 25 mM Tris buffer to reduce sample turbidity. The desired amount of Plasmodium falciparum lactate dehydrogenase (P<DH) was added to 1 ml of a diluted solution of the sample to obtain analyte concentrations ranging from 1 fM to 1 μm (based on the undiluted blood). The functionalised substrates were incubated with 1 ml of contaminated blood solution for 2 hours at room temperature.

7. washing as above
8. fluorescence aptamer-based assay

5-FAM and Cy-5 labelled malaria aptamers were transferred at a 1:1 ratio to 1 ml of 10 mM phosphate buffered saline (PBS) to obtain an aptamer concentration of 0.1 μM (i.e. the Malaria2008 aptamer with fluorophore bound to it). The solution was gently shaken in the absence of light for 2 hours using a rocking shaker. The result was a sandwich arrangement. Such an arrangement is also exemplary shown in FIG. 6.

9. washing as above
10. measurement of the fluorescence signal

Fluorescence images were with a Zeiss Axio Observer Z1 phase-inverted contrast fluorescence microscope equipped with Zeiss Colibri.2 LED light source (modules 470 and 625 nm), Zeiss Plan-Apochromat lens 10×/0.45 Ph 1 M27 (FWD=2.1 mm), 38 HE filter (excutation 450-490 nm/emission 500-550 nm) and Kubus 50 Cy5 filter (excitation 625-655 nm/emission 665-715 nm) and pco.edge 5.5 sCMOS photodetector (scale 0.650 μm×0.650 μm per pixel, image size 2560×2160 pixels, scaled image size 1.66 mm×1.40 mm, 16 bit dynamic range, 2 seconds exposure time for each image). The taken fluorescence images were processed using ImageJ software to process the full intensity emanating from the bright spots. First, the RGB images were split into two channels containing the red and green components to analyse the portions of the two fluorophores separately. Again, the “rolling ball” algorithm was used. FIG. 7 shows the resulting image in which the red and green channels are shown reunited (shown here in black and white for reproduction purposes).

11. testing of the specificity

The specificity of the apta immunosensor was tested against lactate dehydrogenase of Plasmodium vivax (P<LDH), which is 90% identical to P<LDH. The P<LDH was added to uninfected human blood (diluted 1:100 in 1 mL 25 mM Tris buffer) to obtain the highest analyte concentration tested in the calibration curve (1 μM based on undiluted whole blood). The measurement of the fluorescence intensity showed that although the lower bioreceptor layer—consisting of pan-malaria anti-PLDH—can detect any Plasmodium malaria marker, in the case of P<LDH no significant cross-reaction could be detected due to the extremely high specificity of the aptamers used as the upper bioreceptor layer, but the fluorescence for the desired target (P<LDH) was orders of magnitude higher, thus a very high specificity was proven.

1.-14. (canceled)

15. A biomarker detection sensor for detection of disease-specific biomarkers comprising a substrate, the substrate having bound thereon
metal nanoparticles arranged in branched two-dimensional structures and/or a two-dimensional lattice, and
anti-biomarker receptors bound to the metal nanoparticles.

16. The biomarker detection sensor according to claim 15, wherein the metal nanoparticles are gold nanoparticles.

17. The biomarker detection sensor according to claim 15, wherein the disease-specific biomarkers comprise PfLDH and the anti-biomarker receptors comprise anti-pLDH antibodies pre-treated with UV radiation.

18. The biomarker detection sensor according to claim 15, wherein a ratio R of diameter D of the metal nanoparticles to a distance d between individual metal nanoparticles in the two-dimensional lattice is >2.3.

19. The biomarker detection sensor according to claim 15, wherein
a) the metal nanoparticles
a1) are hexagonally or hexagonally-close packed,
or
a2) are arranged in branched band-like structures,
or
a3) form a combined two-dimensional structure wherein parts of the structure are hexagonally or hexagonally-close packed, and parts of the structure are arranged in branched, band-like structures;
b) a diameter D of the metal nanoparticles is between 40 and 70 nm, and
c) a distance K between individual metal nanoparticles is between 16 and 24 nm.

20. A biomarker detection sensor kit comprising
A) at least one biomarker detection sensor according to claim 15,
B0) a preparation comprising at least one biomarker-specific aptamer having a fluorophore bound thereto,
optionally
B1) at least one further preparation comprising at least one identical biomarker-specific aptamer with a different fluorophore bound thereto,
and/or
B2) at least one further preparation comprising at least one other biomarker-specific aptamer having another fluorophore bound thereto.

21. The biomarker detection sensor kit according to claim 20, wherein B2) is selected from
B2i) at least one other biomarker-specific aptamer for detection of the same biomarker but binding to a different epitope of the biomarker with a different fluorophore bound thereto,
and/or
B2ii) at least one other biomarker-specific aptamer for detection of a further biomarker with a different fluorophore bound thereto.

22. The biomarker detection sensor kit according to claim 20, wherein in BO) the at least one biomarker-specific aptamer comprises a malaria 2008 aptamer.

23. The biomarker detection sensor kit of claim 22, further comprising cyanine 5 bound to the malaria 2008 aptamer as a fluorophore.

24. The biomarker detection sensor kit according to claim 20, wherein
in B0) the preparation comprises 5-FAM fluorophore bound to the at least one biomarker-specific aptamer, and
in B1) the at least one further preparation comprises Cy5-fluorophore bound to the at least one identical biomarker-specific aptamer.

25. A method for determination of disease-specific biomarkers in body fluids comprising
i) providing the biomarker detection sensor according to claim 15,
ii) applying to the sensor
iia1) a body fluid sample, and
iia2) at least one biomarker-specific aptamer having a fluorophore coupled thereto either after iia1) or simultaneously with iia1), or
iib) a mixture of body fluid sample and at least one biomarker-specific aptamer with fluorophore coupled thereto,
iii) irradiating the sample applied to the sensor with light source,
iv) detecting fluorescence emission from the sample,
v) optionally analysing the fluorescence emission, and
vi) optionally storing and/or displaying the analysed fluorescence emission.

26. The method according to claim 25, wherein the body fluid sample is a blood sample.

27. The method according to claim 25, wherein the anti-biomarker receptors of the biomarker detection sensor comprise anti-pLDH antibodies and the at least one biomarker-specific aptamer comprises malaria-2008 aptamer.

28. The biomarker detection sensor of claim 15, being for qualitative or quantitative determination of disease-specific biomarkers.

29. The biomarker detection sensor of claim 28, wherein the biomarkers comprise malaria biomarkers in blood fluid samples.

30. The biomarker detection sensor kit of claim 20, being for qualitative or quantitative determination of disease-specific biomarkers.

31. The biomarker detection sensor kit of claim 30, wherein the biomarkers comprise malaria biomarkers in body fluid samples.

32. The method according to claim 25, being for qualitative or quantitative determination of disease-specific biomarkers.

33. The method according to claim 32, wherein the biomarkers comprise malaria biomarkers.

34. The biomarker detection sensor of claim 15, wherein the substrate is a substrate of an automated multititer plate.
